(19) United States
(12) Patent Application Publication
(10) Pub. No.: US 2013/0098363 A1
(43) Pub. Date: Apr. 25, 2013

(54) ENDOTRACHEAL CUFF PRESSURE REGULATION CIRCUIT AND METHOD

(71) Applicants: Vito Forte, Toronto (CA); Joseph Fisher, Thornhill (CA)

(72) Inventors: Vito Forte, Toronto (CA); Joseph Fisher, Thornhill (CA)

(21) Appl. No.: 13/695,318

(22) PCT Filed: May 2, 2011
(86) PCT No.: PCT/CA11/00506

§ 371 Date: Jan. 10, 2013

Related U.S. Application Data
(60) Provisional application No. 61/329,776, filed on Apr. 30, 2010.

(51) Int. Cl. A61M 16/04

(52) U.S. Cl. CPC A61M 16/0434
USPC 128/204.23

(57) ABSTRACT

Cuff pressure modulation results in decreased severity of injury to the subglottic region and upper trachea. A simple device is capable of modulating the pressure in the cuff of a regular endotracheal tube, by coordinating the pressure level to be maximal during the inspiratory phase and minimal during the expiratory phase. This allowed for regular positive airway pressure ventilation as during inspiration the seal was maintained between the ETT and the tracheal mucosa by the inflated cuff, but during expiration cuff deflation allowed the cuff pressure to drop in the subglottic and tracheal area.

### FIELD OF THE INVENTION

The present invention relates to a method and apparatus for preventing ischemic tracheal mucosal damage during intubation.

### BACKGROUND OF THE INVENTION

Intubation with an endotracheal tube (ETT) is an effective method for mechanical ventilation, in both adults and children. However, endotracheal tube-related laryngotracheal injury is a well-recognized potential complication.¹⁻³ The major contributor to the development of airway injury is the pressure that the ETT exerts at points of contact with the laryngotracheal mucosa, potentially leading to ischemic necrosis⁴. Mucosal damage and inflammation in the trachea can be demonstrated even after short periods of intubation.^{5,6}

In adults, high volume low-pressure cuffs have decreased the incidence of ETT-related mucosal damage and subglottic stenosis. However, an ETT cuff pressure exceeding capillary perfusion pressure may result in impaired mucosal blood flow, thereby significantly contributing to the tracheal morbidity associated with intubation.³ In the pediatric population, long-term ventilation using uncuffed ETTs has long been recognized to have the potential to cause severe subglottic stenosis.⁷ Traditional teaching has recommended uncuffed ETTs in children under 8 years of age to reduce the risk of laryngotracheal injury and acceptance of a leak during positive pressure ventilation of 15-20 cm of water.

More recently however, a vivid debate has surfaced about the pros and cons of using cuffed ETTs in children.⁸ Cuffed ETTs have been shown to decrease the number of laryngoscopies and ETT passages through the glottis, reduce the risk of aspiration, and improve precision of end-tidal carbon dioxide monitoring, while not causing an increase in post-intubation stridor.⁹⁻¹³ Used correctly, cuffed tubes have the additional advantages of allowing to seal the trachea as opposed to the cricoid area, allow the use of low to minimal fresh gas flow, accurate pulmonary function testing, and decreased environmental pollution.^{10,13} Fine and Borland suggested that a cuffed ETT should be the first choice when a tube with an internal diameter of 3.5 mm or greater is selected.¹²

Potential disadvantages of cuffed ETTs include difficulty in determining the correct position and herniation of the cuff, and most importantly, the risk of cuff pressure-related tracheal damage. Recent surveys from the United Kingdom¹⁴ and France¹⁵ demonstrated that a minority of anesthetists and pediatric intensive care physicians were routinely employing cuffed tubes for intubation in children, predominantly because of concerns about cuff-related tracheal injuries. The pathological process of cuff-induced stenosis is thought to begin with pressure on the laryngotracheal mucosa, especially when the cuff is over-inflated, resulting in impaired tracheal mucosal blood flow, edema and ischemic necrosis, and eventually formation of fibrotic scar tissue. Unfortunately, no studies have been effectively designed to prospectively compare the incidence of subglottic stenosis between children intubated with cuffed or uncuffed endotracheal tubes.

Developing a mechanism to significantly reduce cuff-related tracheal injuries could result in major benefits for the pediatric population and a more widespread use of cuffed ETTs. It would also be beneficial in reducing the risk of intubation-related injury in older children and adult patients for whom cuffed tubes are the only available option. Attempts to reduce cuff-related injuries by automated maintenance of a constant cuff pressure have failed to reduce tracheal injury in an animal model.¹⁶

### SUMMARY OF THE INVENTION

The present invention is directed a method and device for mitigating endotracheal tube-related injury as well as a breathing circuit incorporating the device including components adapted for this purpose.

According to one aspect, the invention is directed to a device for mitigating endotracheal tube related laryngotracheal injury associated with intubating a patient, and preventing the aspiration into the trachea and lung of potentially infected secretions from the oropharynx to prevent lung infection, the device adapted for use with a mechanical ventilator, and an endotracheal tube of the type having an inflatable endotracheal cuff, the device comprising:
A ventilator port;
An endotracheal tube port;
An air conduit portion fluidly connected to the ventilator port and the endotracheal tube port, the air conduit portion defining at least one airflow path between the ventilator port and the endotracheal tube port;
At least one pressure difference generator operatively associated with the air conduit portion for at least transiently generating a pressure difference between a first pressure region of the airflow path on a ventilator side of the pressure difference generator and a second air pressure region of the airflow path on an endotracheal tube side of the pressure difference generator;
A cuff port for fluidly connecting the first pressure region and the interior of the cuff such that a first pressure in the first pressure region of the at least one airflow path substantially determines the air pressure in the interior of the cuff whereby the cuff pressure is adapted to be reduced in tandem with a ventilator pressure set for an expiratory phase of a breath.

The invention provides parameters for mitigating endotracheal tube related laryngotracheal injury associated with intubating a patient, and preventing the aspiration into the trachea and lung of potentially infected secretions from the oropharynx to prevent lung infection thereby providing for the demarcation of selectable ventilator settings and suitable pressure differences to be effected by a pressure difference generator.

The pressure difference generated by the at least one pressure difference generator determines, in cooperation with a ventilator pressure setting (e.g. suitable to prevent tracheal injury and provide suitable inspiratory pressures and PEEP), relative first and second pressures in the first and second pressure regions, respectively, and wherein the first pressure and the second pressure cooperate to inhibit fluid movement around the outside of the cuff when the cuff is inflated to respective differing first pressures. The differing first pressures correspond to ventilator pressure settings for an inspiratory phase of a breath and an expiratory phase of the breath, respectively. The differing first pressures optionally include a range of differing inspiratory pressures, for the inspiratory phase of a breath and optionally at least one expiratory phase pressure, optionally a range of different expiratory pressures, the expiratory phase pressure(s) corresponding to one or more useful positive end expiratory pressure(s).

Optionally, the pressure difference generator divides the first pressure region and second pressure region.

Optionally, the pressure difference generator is a valve that opens toward the endotracheal tube at a predetermined pressure in the first pressure region in response to a ventilator pressure generated by the ventilator for an inspiratory phase of a breath.

Optionally, the pressure difference generator is a valve that opens toward the ventilator at a predetermined pressure in response to a second pressure in the second pressure region. Optionally, the pressure difference generator opens at a pressure that is greater than a nominal opening pressure for example an opening pressure that generates positive end expiratory pressure (PEEP).

Optionally, the air resistance component is a bi-directional valve assembly including a first closure assembly that open towards the ventilator responsive to exhalation pressure generated in the second pressure region during an expiratory phase of a breath (defining an expiratory valve), and a second closure assembly that generates a pressure difference between the first pressure region and the second pressure, wherein first pressure is greater than the second pressure; the first pressure optionally corresponding to a cuff pressure which exceeds the second pressure (the airway pressure) by an amount sufficient to prevent substantial air leakage or fluid leakage around the cuff at a pre-determined range of peak inspiratory pressures generated by the ventilator. Optionally, the first closure assembly comprises a valve seat and a valve closure member, for example, an expiratory valve flap. The valve closure member, optionally, an expiratory valve flap, is optionally adapted e.g. sufficiently rigid, to provide a desired positive end expiratory pressure (PEEP). Optionally, the second closure assembly comprises a valve seat and a second closure element that is movable between a closed position in which it sealingly engages the valve seat and an open position in which it is spaced from valve seat. The second closure element is normally in a closed position, and is optionally operatively associated with a biasing means, for example a spring, that determines the opening pressure of valve closure member.

Optionally, air conduit portion defines two airflow paths between the ventilator port and the endotracheal tube port. An expiratory valve may be operatively associated with a first airflow path and a valve providing a second closure assembly with a second airflow path.

According to another aspect the invention is directed to a device for mitigating endotracheal tube related laryngotracheal injury associated with intubating a patient, and preventing the aspiration into the trachea and lung of potentially infected secretions from the oropharynx to prevent lung infection, the device adapted for use with a ventilator, and an endotracheal tube of the type having an inflatable cuff, the device comprising an inflatable cuff port and an air conduit portion including:
a) a first portion which is: (1) configured in a Y shape for fluidly joining an expiratory limb and an inspiratory limb of a ventilator breathing circuit; or (2) adapted to be connected to a Y connector which fluidly joins the expiratory limb and the inspiratory limb of a ventilator breathing circuit;
b) a second portion that is fluidly connected to or fluidly connectable to an endotracheal tube; and
c) a third portion positioned between the first portion and the second portion, the third portion fluidly connected to the cuff port such that the air pressure in at least the third portion of the air conduit portion substantially determines the air pressure in the interior of the inflatable cuff and enables the cuff pressure to be reduced in tandem with a lower ventilator pressure set for an expiratory phase of a breath.

Optionally, the aforesaid further comprises at least one pressure difference generator (optionally in the form of an airflow resistance element) that is operatively associated with the third portion for at least transiently generating a pressure difference between a first pressure region of the third portion on a ventilator side of pressure difference generator and a second air pressure region of the third portion on an endotracheal tube side of the pressure difference generator, the cuff port positioned in the first air pressure region of the third portion such that the pressure in the first pressure region of the third portion is capable of substantially determining the air pressure in the interior of the cuff.

Embodiments of the invention described herein as applicable to a particular aspect of the invention are to be generally understood (unless the context dictates otherwise) as being applicable to the aforesaid aspects and all other aspects of invention and vice versa. The device as aforesaid optionally further comprise other ventilator breathing circuit elements including a Wye connector, inspiratory and expiratory tubing and a cuffed endotracheal tube. According to a related aspect the invention is directed to a kit comprising one or more components of such a breathing circuit including the devices as aforesaid.

According to another aspect, the invention is directed to a method for mitigating endotracheal tube related laryngotracheal injury associated with intubating a ventilated patient (including a patient undergoing anesthesia) with an endotracheal tube of the type having an inflatable cuff, comprising the step of reducing the cuff pressure against the laryngotracheal mucosa to between 1 and 5 cm H₂O during exhalation phases of the patients breathing cycles.

Optionally, the cuff pressure is reduced to between 2 and 4 cm H₂O during exhalation, more preferably approximately 2 or 3 cm H₂O. However, it will be appreciated that the preferred parameters are not limiting and the method may be implemented by setting the cuff pressure to accord with the ventilator setting upon expiration provided the PEEP pressure is less than 20 cm of water.

Optionally, the method is accomplished by independently re-setting the cuff pressure during exhalation to be in the desired pressure range of approximately 2 to 4 cm H₂O, optionally 2 to 3 cm H₂O, for example at all times similar to that of the airway pressure generated by the ventilator. This may be accomplished electronically, for example, using a separate cuff air pressure generator, or mechanically by equilibrating the pressure in the cuff with the airway pressure in a portion of the breathing circuit proximal to the ventilator. Optionally, the cuff pressure is maintained at the desired value during exhalation by setting the ventilator to generate a suitable positive end expiratory pressure (PEEP) for the patient during exhalation. Optionally the PEEP is set at 2 to 4 cm H₂O and the cuff pressure is dictated by the PEEP pressure insofar as patient airway pressure on the outside of the cuff during exhalation does not exceed this pressure. The term “equilibrate” or “equilibration” means that the inflatable reservoir in the cuff pressure is fluidically connected to the conduit carrying air away from the ventilator and affected by its pressure at least insofar as it is not subsequently adjusted. As described hereafter, the invention herein obviates the need for such adjustment and provides a simple device that can be retrofitted to any existing endotracheal tube (ETT) and associated breathing circuit.

Optionally, the method comprises setting the cuff pressure to be higher than the patient airway pressure during inspiration by interposing a valve, optionally a PEEP valve (for example having an opening pressure of 5 cm H₂O), between a first portion of the ventilator breathing circuit proximal to the ventilator—having the highest pressure in the breathing circuit (wherein there is a port leading to the cuff) and the portion of the breathing circuit proximal to the endotracheal tube, having a lower air pressure attributable to the valve. This valve may be a bidirectional valve which includes an expiratory valve.

The term “ventilator” encompasses any mechanical apparatus that creates positive airway pressure that is differentially geared to inspiratory and expiratory phases of breathing and suitable for use with an endotracheal tube.

According to another aspect the invention is directed to a device for use with a ventilator and an endotracheal tube of the type having an inflatable cuff, comprising:

one or more airflow path defining components that define at least one airflow path between a port leading to the ventilator and a port leading to the endotracheal tube;

at least one pressure differential generating component for creating a pressure differential between the port leading to the ventilator and the port leading to the endotracheal tube, the pressure differential constituted at least in part by a higher first pressure in a first portion of the device proximal to the port leading to the ventilator, the first pressure dictated at least in part by the air pressure generated by the ventilator, and a lower second pressure in a second portion of the device proximal to the endotracheal tube; and

a port in the first portion of the device for fluidically connecting the first portion of the device proximal to the inflatable cuff, whereby the pressure in the cuff is dictated at least in part by the air pressure in the first portion of the device.

Optionally, the respective ports leading to the ventilator and endotracheal tube are adapted for direct attachment to standard configurations of breathing circuit elements associated with the ventilator and the endotracheal tubes (i.e. their mating ends), obviating the need for special adaptors to facilitate mating the respective ends of these various components.

Optionally, the pressure differential generating component comprises a valve positioned between the first portion of the device and the second portion of the device, the valve having an opening pressure that at least in part dictates the pressure differential between the first portion of the device and the second portion of the device, for example, a valve having an opening pressure of approximately 3 to 7 cm of H₂O, optionally 5 cm of H₂O. Optionally, the valve is a PEEP valve including a biasing means for setting the pressure, the biasing means optionally a spring. Optionally, the pressure differential generating component is a bidirectional valve which integrates (1) a valve having an opening pressure that at least in part dictates the pressure differential between the first portion of the device and the second portion of the device, and (2) a one way expiratory valve. Alternatively, the first portion of the device and the second portion of the device are connected by two airflow paths, an inspiratory first air flow path allocated to the pressure differential generating component and an expiratory second air flow path comprising a one way expiratory valve.

The invention is also directed to the use of a device as previously defined but without a port in the first portion of the device for fluidically connecting the first portion of the circuit to the inflatable cuff, wherein the use is for connection to a ventilator breathing circuit that does have such a port, as well as to a kit comprising the last mentioned device and a breathing circuit components that do have this port. The invention is also directed to the use of the aforesaid devices or kit for mitigating or preventing larygiotracheal mucosal tissue injury.

Optionally, the device is constituted in a single principal component or body. Therefore, according to another aspect the invention is directed to a device for use with a ventilator and an endotracheal tube of the type having an inflatable cuff, comprising:

a body portion including a plurality of ports that define at least one airflow path between a first port leading to the ventilator and a second port leading to the endotracheal tube;

at least one pressure differential generating valve for creating a pressure differential between the first port and the second port, the pressure differential dictated at least in part by an opening pressure of the valve which translates into a higher first pressure in a first portion of the device on a side of the valve proximal to the first port, and a lower second pressure in a second portion of the device on the other side of the valve proximal to the second port;

a third port in the first portion of the device for fluidically connecting the first portion of the device to the inflatable cuff, whereby the pressure in the cuff is dictated at least in part by the air pressure in the first portion of the device.

Optionally the aforesaid device comprises a one way expiratory valve which only opens to allow air flow towards the first portion of the device. This expiratory valve is optionally integrated within the pressure differential generating valve to form a bidirectional valve, namely a valve which resists flow in one both directions in the absence of each respective valve-opening pressure acting on the valve, which in a preferred embodiment are different pressures as described below.

According to another aspect, the invention is directed to a breathing circuit assembly, for use with a ventilator and an endotracheal tube of the type having an inflatable cuff, comprising:

one or more airflow path defining components that define at least one airflow path between a port leading to the ventilator and a port leading to the endotracheal tube;

at least one pressure differential generating component for creating a pressure differential between the port leading to the ventilator and the port leading to the endotracheal tube, the pressure differential constituted at least in part by a higher first pressure in a first portion of the circuit proximal to the port leading to the ventilator, the first pressure dictated at least in part by the air pressure generated by the ventilator, and a lower second pressure in a second portion of the circuit proximal to the endotracheal tube; and

a port in the first portion of the circuit for fluidically connecting the first portion of the circuit to the inflatable cuff, whereby the pressure in the cuff is dictated at least in part by the air pressure in the first portion of the circuit.

Similarly, the pressure differential generating component may comprises a valve positioned between the first portion of the circuit and the second portion of the circuit, the valve having an opening pressure that at least in part dictates the pressure differential between the first portion of the circuit and the second portion of the circuit, for example, a PEEP-like valve including a biasing means. The valve may have an opening pressure of approximately 5 cm of H₂O. Similarly, the pressure differential generating valve may be a bidirectional valve which integrates a valve having an opening pressure that at least in part dictates the pressure differential between the first portion of the circuit and the second portion of the circuit and a one way expiratory valve. Alternatively, the first portion of the circuit and the second portion of the circuit are connected by two airflow paths, an inspiratory first air flow path allocated to the pressure differential generating component and an expiratory second airflow path comprising a one way expiratory valve.

The term “standard” used with reference to an endotracheal tube or other breathing circuit elements includes components with mating ends that become the standard or one of the standards at any given time.

According to another aspect, the invention is directed to a device for use with a ventilator, and an endotracheal tube of the type having an inflatable cuff, comprising:

A ventilator port;

An endotracheal tube port;

An air conduit portion fluidly connected to the ventilator port and the endotracheal tube port, the air conduit portion defining at least one airflow path between the ventilator port and the endotracheal tube port;

A cuff port operatively associated with the air conduit portion for fluidly connecting the at least one airflow path and the interior of the cuff such that the pressure in the airflow path substantially determines the air pressure in the interior of the cuff and the cuff pressure is reduced in tandem with a lower ventilator pressure set for the expiratory phase of a breath.

Optionally, at least one air resistance component is operatively associated with the air conduit portion for dividing, and generating a pressure difference between, a first pressure region of the airflow path on a ventilator side of air resistance component and a second air pressure region of the airflow path on an endotracheal tube side of air resistance component, the cuff port positioned in the first air pressure region of the at least one air flow path such that the pressure in the first pressure region of the airflow path substantially determines the air pressure in the interior of the cuff.

Optionally, the amount of air resistance generated by the at least one air resistance component is pre-selected to determine a relative second pressure in the second pressure region such that the first pressure and second pressure cooperate to inhibit fluid movement around the outside of the cuff over the course of a breath when the cuff is inflated to respective differing first pressures.

According to another aspect the invention is directed to a method for mitigating endotracheal tube related laryngotracheal injury associated with intubating a patient and preventing the aspiration into the trachea and lung of potentially infected secretions from the oropharynx to prevent lung infection, with an endotracheal tube of the type having an inflatable cuff, the method comprising the step of reducing cuff pressure against the laryngotracheal mucosa to between 3 and 19 cm H₂O during an expiratory phase of the patient's breathing cycles.

The cuff pressure against the laryngotracheal mucosa during an expiratory phase of the patient's breathing cycles is substantially determined by a ventilator pressure setting set for the expiratory phase of the patient's breathing cycles, optionally by setting the PEEP setting on the ventilator to between 3 and 19 cm H₂O.

Optionally, the cuff pressure is equilibrated with the ventilator pressure setting by organizing the airflow to the cuff to be channeled to the cuff from an airflow path between the ventilator and the endotracheal tube, the airflow path fluidly connected to the interior of the cuff via a cuff port.

Optionally, the cuff pressure is organized to be different than the patient's airway pressure during an inspiratory phase of the patient's breathing cycles.

Optionally, the cuff pressure is organized to be different than the patient's airway pressure during an expiratory phase of the patient's breathing cycles.

Optionally, the patient's airway pressure is organized to be less the cuff pressure by interposing a pressure difference generator in the airflow path between the endotracheal tube and the cuff port, the pressure difference generator at least transiently generating a pressure difference between a first pressure region of the airflow path on a ventilator side of the pressure difference generator and a second air pressure region of the airflow path on an endotracheal tube side of pressure difference generator.

### BRIEF DESCRIPTION OF THE DRAWINGS

FIG. 1 is a schematic representation of one embodiment of a device according to the invention.

FIG. 1a is a sectional view along line 1a showing a concentric relationship between the different parts according to one embodiment of the device.

FIG. 2 is a schematic diagram of a preferred embodiment of the device connected on one side to an endotracheal tube having a cuff and on the other side to a portion of a breathing circuit leading to the ventilator.

FIG. 2a is a schematic diagram of an alternative embodiment of the device according to invention wherein two examples of suitable pressure difference generators are allocated to two different airflow paths within the device.

FIG. 3 is a schematic diagram of one embodiment of a breathing circuit comprising the device, with the device shown in an inspiratory mode, the device connected on one side to an endotracheal tube having an inflatable cuff and on the other side to a portion of a breathing circuit leading to the ventilator, and also showing the endotracheal tube fitted within a schematic representation of a portion of a patient's trachea.

FIG. 4 is a schematic diagram of a preferred embodiment of a breathing circuit comprising the device, the device shown in an expiratory mode, the device connected on one side to an endotracheal tube having an inflatable cuff and on the other side a portion of a breathing circuit leading to the ventilator.

FIG. 5 is pressure tracing showing relative pressures in an inflatable cuff and in patient subject airway showing a consistently higher pressure in the cuff.

FIG. 6 is an axial microscopic section of the upper trachea from an animal that was ventilated for four hours with constant cuff inflation pressure. The section demonstrates significant epithelial loss, extensive subepithelial and glandular necrosis, and acute inflammation (hematoxylin-eosin, magnification ×100).

FIG. 7 is an axial microscopic section of the upper trachea from an animal that was ventilated for four hours using modulated cuff inflation pressure according to a method of the invention. The section demonstrates mainly superficial damage, such as epithelial compression and loss, with normal subepithelial and glandular layers (hematoxylin-eosin, magnification ×100).

FIG. 8a is a table (Table 1) presenting a grading scale for describing the severity of laryngotracheal injury¹⁷

FIG. 8b is a table (Table 2) comparing scores for various categories of histopathological injury to accompany a grading scale for describing the severity of laryngotracheal injury.¹⁷

FIG. 9 is a table (Table 3) comparing baseline physiological characteristics of the two animal study groups in which the effects of cuff pressure were tested

FIG. 10 is a schematic representation of an alternative cuff reducing pressure scheme described in Example 1 used to generate data on the effects of cuff pressure on the severity of laryngotracheal injury.

FIG. 11 is a schematic diagram of a preferred embodiment of a breathing circuit comprising the device, the device shown in an expiratory mode, the device connected on one side to an endotracheal tube having an inflatable cuff and on the other side a portion of a breathing circuit leading to the ventilator.

FIG. 12 is a schematic diagram of a preferred embodiment of a breathing circuit comprising the device, the device connected on one side to an endotracheal tube having an inflatable cuff and on the other side to a breathing circuit leading to the ventilator.

### DETAILED DESCRIPTION OF THE INVENTION

In one embodiment, the present invention is directed to a device that is adapted to fluidly connect the interior of the endotracheal cuff to an air conduit portion of the device which receives airflow from the ventilator and hence is at ventilator pressure. The endotracheal cuff may be consistently inflated to mechanical ventilator pressures including the lower pressures set for the expiratory phase of a breath. For the inspiratory phase of breath, the cuff pressure may also be set to exceed airway pressure to inhibit fluid (gas or liquid) movement around the outside of the endotracheal cuff. Preferably, a pressure difference generator is used to lower airway pressure relative to cuff pressure on inspiration. On expiration, a pressure difference generator may be used to generate PEEP or add to the PEEP generated by a mechanical ventilator. The PEEP generated by a mechanical ventilator controls the cuff pressure. Hydrostatic pressure of fluid sitting against the cuff may be in the order of 2 or 3 cm of water and cuff pressure should prevent this fluid from leaking down. Airway pressure serves this purpose as well during expiration. However, insufficient cuff pressure may dissipate airway pressure so at lower cuff pressures in which the benefit of friction resulting from the cuff pressure is reduced, the cuff pressure preferably exceeds the hydrostatic pressure since the lung pressure tends to equilibrate to the cuff pressure once the lung pressure goes down to the PEEP. Since the cuff pressure is dictated by the ventilator PEEP, excess PEEP supplied by the expiratory valve might be counterproductive because this PEEP contributes to airway pressure but does not contribute to cuff pressure.

The term “endotracheal tube port” means an opening of a size suitable size to channel the flow of a gas to or via an endotracheal tube to and from a patient. Such a port may conventionally be designed to receive a conventional endotracheal tube but could also be implemented within a male connector and with any device that functions as an endotracheal tube using an inflatable means to effect a seal in a patient airway.

The term “ventilator port” means an opening leading to/from a ventilator of a size suitable to channel the flow of a gas via a gas conduit leading from a ventilator to an endotracheal tube, such conduits conventionally in the form of connectors and conventional tubing used with a ventilator. For example, a suitable connector designed for use with a ventilator breathing circuit, such as a Wye connector may be fluidly connected to a device of the invention via the “ventilator port”. Such a port may conventionally be designed to receive a Wye connector but could also be implemented within a male connector portion.

The term “exhalation pressure” means the pressure generated by the lung in the course of exhalation with or without mechanical assistance.

The term “expiratory valve” means a valve that, in use, opens away from the patient responsive to exhalation pressure, for example pressure generated in the second pressure region during an expiratory phase of a breath

The term “incremental cuff pressure” means, in relation to an inspiratory phase of breath, a pressure greater than the airway pressure that is empirically determined to be sufficient to prevent leakage in an amount that compromises a positive pressure ventilation regimen and fluid leakage leading to undesirable aspiration of fluid. The effect of friction of the cuff against the trachea may minimize the incremental cuff pressure at higher inspiratory pressure. The effect of friction will also prevent dissipation of airway pressure via the endotracheal cuff during expiration. Therefore, it is understood that the invention is not limited by selecting values for variables described herein that are obviated by the benefits of friction. Hence, the choice of pressure difference generator will be dependent on cuff pressure and choice of cuff pressure when the benefits of friction are added will impact on the choice and necessity for a pressure difference generator.

With respect to an expiratory phase of a breath, the cuff pressure may be equal to or less than the airway pressure and still be sufficiently high when in excess of 2 or 3 cm of water to prevent fluid leakage leading to an undesirable aspiration of fluid.

The term “breath” refers to one inspiratory phase and an ensuing expiratory phase of a breath.

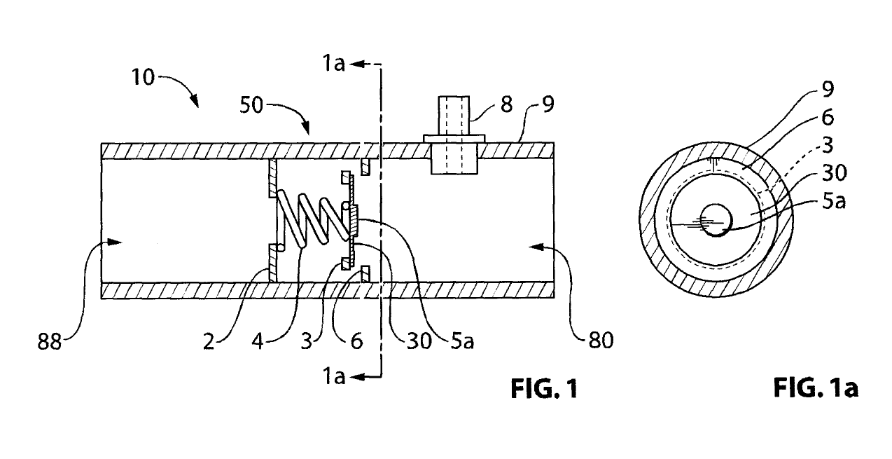

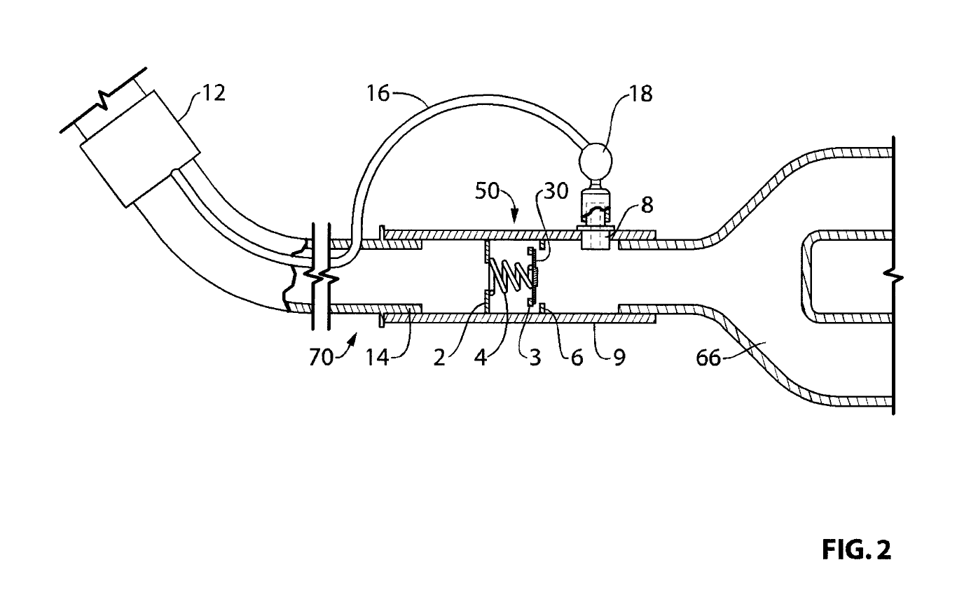

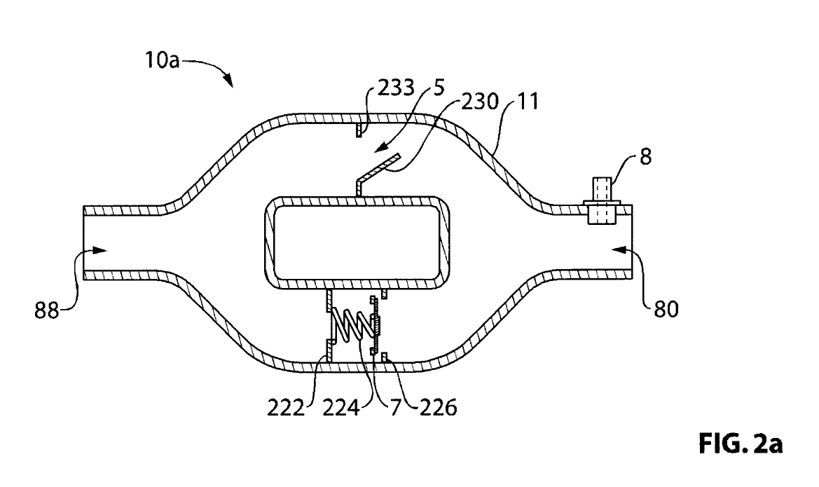

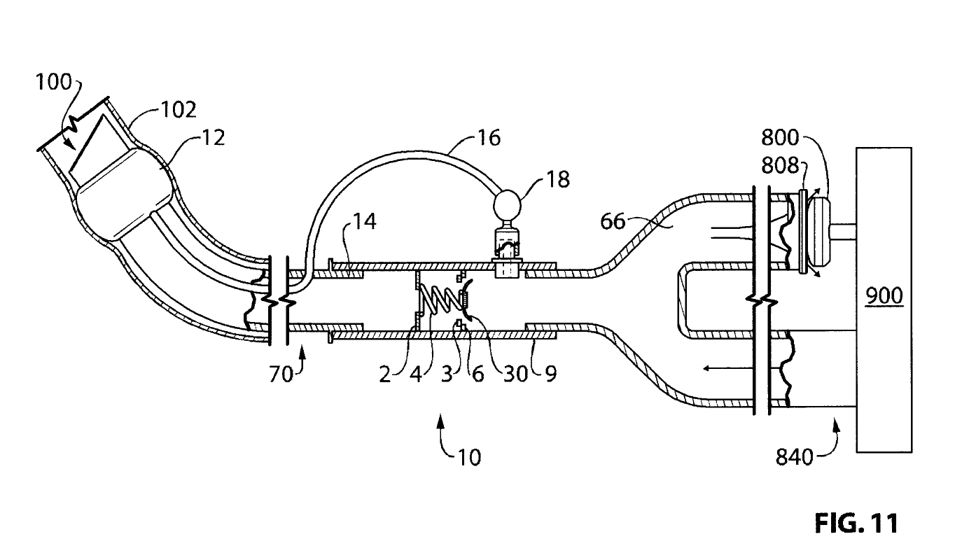

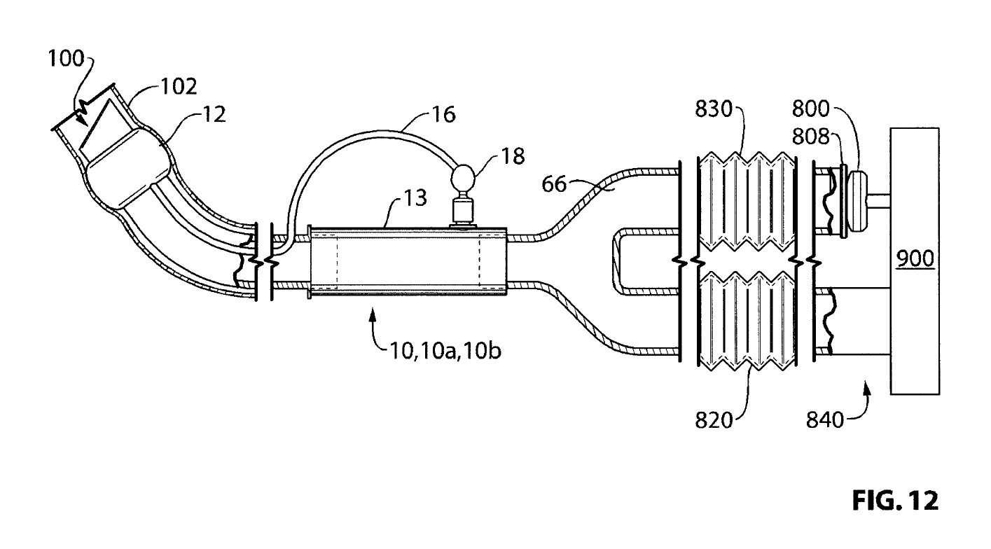

As shown in FIGS. 1 and 2, in one embodiment of a device according to the invention, the device 10 comprises an air conduit portion 9 extending between a ventilator port 80 and an endotracheal tube port 88 and optionally at least one pressure difference generator, optionally in the form of a bidirectional valve 50 which combines an “expiratory valve” that opens toward the ventilator, typically having an opening pressure of 1 to 2 cm H₂O and a valve that resists airflow from the ventilator to the endotracheal tube 70 to generate a pressure difference. Optionally at least in part due it's opening pressure, for example an opening pressure of 5 cm H₂O, a pressure difference between the ventilator port 80 leading to the ventilator and the endotracheal tube port 88 is generated. The pressure difference in this embodiment is constituted at least in part by a higher first pressure in a first pressure region of the device proximal to the ventilator port 80 which leads to the ventilator 900 (shown in FIGS. 11 and 12), the first pressure substantially determined by the air pressure generated by the ventilator, and a lower second pressure in a second pressure region of the device proximal to the endotracheal tube 70.

A cuff port 8 in the first pressure region of the device 10 fluidically connects the first pressure region of air conduit portion 9 (11, 13) to the inflatable endotracheal cuff 12, whereby the pressure in the cuff 12 is dictated at least in part by the air pressure in the first pressure region of the air conduit.
In one embodiment of the invention, a bi-directional valve 50 (obtainable from Vital Signs Inc., World Headquarters 20 Campus Road, Totowa, N.J. 07512 or Intersurgical Ltd. Creane House, Molly Millars Lane, Wokingham, Berkshire RG412RZ), comprises a first closure assembly which functions as an expiratory valve and a second closure assembly which is designed in the manner of a PEEP-like valve, the second closure assembly optionally including spring 4, spring retainer 2 and “PEEP-like valve” retainer 6. Flap 30 is shared with the first closure assembly to serve in part as closure member for the second closure assembly. The first closure assembly may be made up of standard parts of an expiratory valve including an expiratory valve retainer 3 and an expiratory flap or disc 30 serving as a closure member.

The term “port” could mean receives or could be understood to be a male connector.

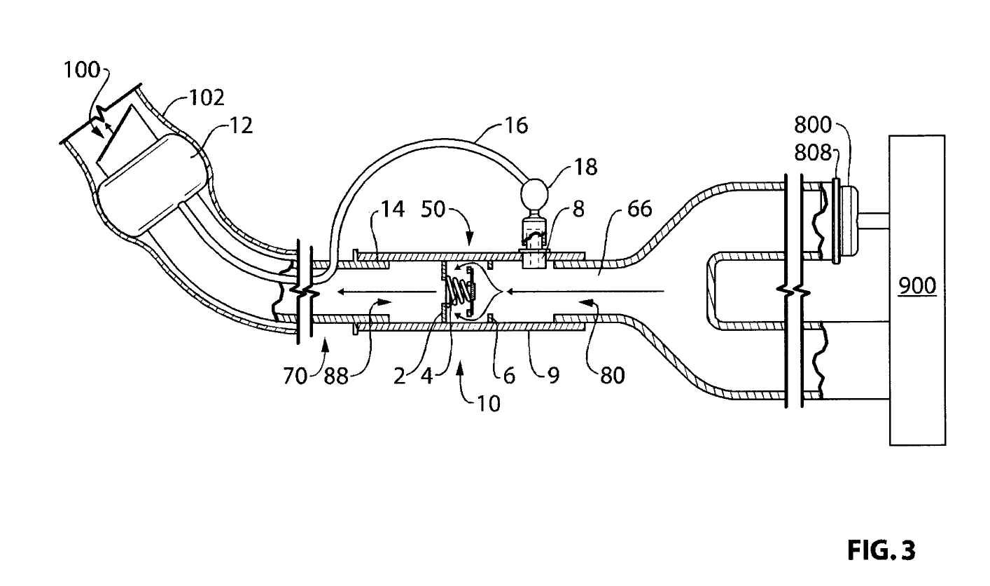

In the usual orientation, the known bi-directional valve 50 shown in FIG. 1 was originally designed to provide PEEP when deployed in the opposite direction than is shown in FIGS. 1 to 4. Notably, the bi-directional valve was not manufactured with a port or fitting 8 for mounting a tube 16 leading to an endotracheal cuff 12. As shown, for example in FIG. 3 and others, cuff tube 16 is operatively connected to balloon 18 and leads to an opening in the endotracheal tube cuff 12. To protect against endotracheal cuff related injury and aspiration, as opposed to providing PEEP, the respective sizes of the ports 80 and 88 on each end of the commercially available bi-directional valve (currently fits the 15 mm ETT connector 14 and Wye connector 66), would have to be reversed. A connection to the ETT cuff pilot tube 16 would have to be built into the device or provided via a separate connector between the device and the Wye, or one would employ a Wye connector with the cuff port fitting 8 e.g. a male luer connector.

As shown in FIG. 2a, an alternate embodiment of the device 10a comprises two airflow pathways and two distinct closure assemblies akin to those of the bidirectional valve 50. One closure assembly is constituted by an expiratory valve 5 which includes flap retainer 233 and valve flap 230. The other closure assembly comprises spring retainer 222, spring 224 and retainers and retainer 226. The closure member 7 may be of any conventional type. The respective closure assemblies are shown to be functionally allocated to two different air flow pathways.

As shown in FIG. 2 when the device 10 is not in use, the expiratory valve disc 30 is pressed against the expiratory valve retainer 3. This is in a sense a floating retainer that is linked to the PEEP spring 4.

As seen in FIG. 3, during inspiration the expiratory valve flap 30 is pressed against the expiratory valve retainer 3 to form a PEEP-like valve closure element. On inspiration, when the airway pressure attributable to the inspiratory pressure set on the ventilator exceeds the PEEP-like valve setting (dictated by spring parameters), the closure member (3, 30) is separated (pushed away) from the retainer 6. The strength of the spring 4 determines the pressure differential across the PEEP-like valve. The same pressure differential is formed between the endotracheal tube cuff and the patient airway. FIG. 3 illustrates how the endotracheal tube cuff 12 sits within the tracheal lumen 100 and pressed against tracheal wall 102. Device 10 is connected on its downstream end to the endotracheal tube 70 via endotracheal tube connector 14 via endotracheal tube port 88 in the device 10. On the upstream side of the device 10, connected to the device via ventilator port 80, are breathing circuit components leading from the ventilator 900 (also seen in FIG. 12), for example, a Wye connector 66. As shown in FIGS. 3 and 12, device 10 (which optionally may be substituted by device 10a—FIG. 2a) is connected to Wye connector 66, which is in turn connected to expiratory limb tubing 830 and inspiratory limb tubing 820 (shown only in FIG. 12). Inspiratory limb tubing 820 may be connected to the ventilator 900 via a connector portion 840 having a suitable port (not shown). Expiratory limb tubing 830 leads to a suitable connector portion supporting valve seat 808 which cooperates with a variable resistance valve that relieves and thereby controls pressure in the circuit. For example, mushroom valve member 800 is used to variably control the pressure in the circuit (e.g. proportional to the extent that it is inflated to allow air to escape from the circuit) for providing PEEP. As shown in FIG. 3, this valve is closed during inspiration and partially open during exhalation (see FIG. 11 which shows gas escaping the circuit through the mushroom valve due to exhaled gas passing through expiratory valve flap 30—shown open).

As shown in FIG. 3, cuff port 8 leading to the endotracheal cuff pilot balloon 18 and then to endotracheal cuff tube 16 and on to the opening in the endotracheal tube cuff (not shown), is located in upstream of bi-directional valve 50 which defines a first pressure region of the device from which the endotracheal cuff 12 “sees” the ventilatory pressure generated by the ventilator 900.

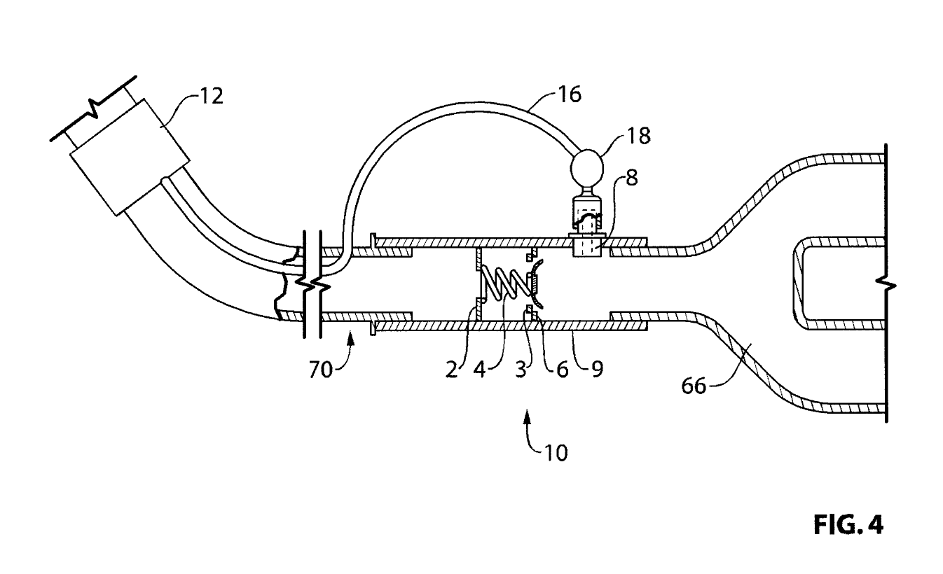

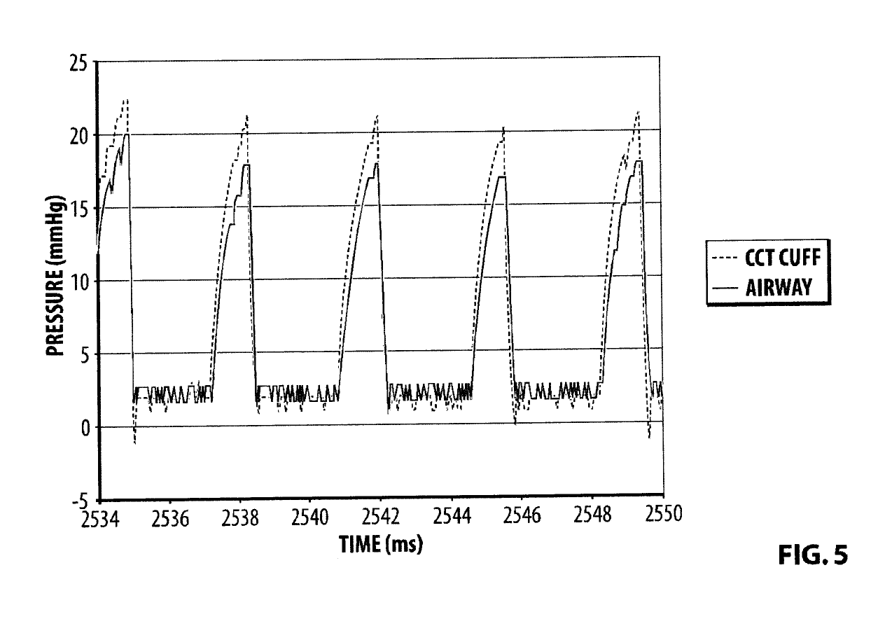

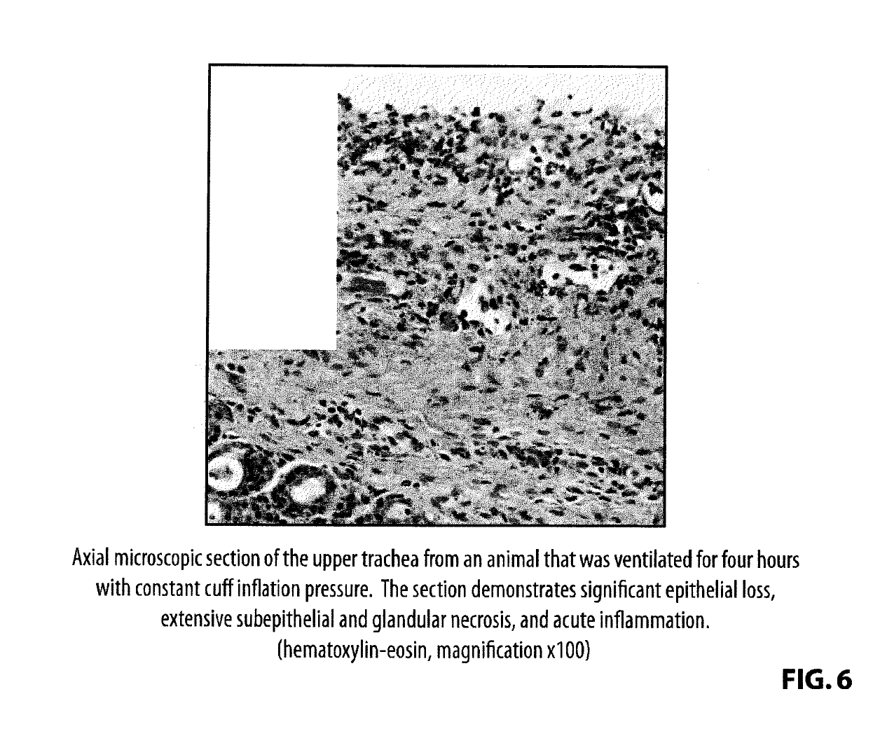

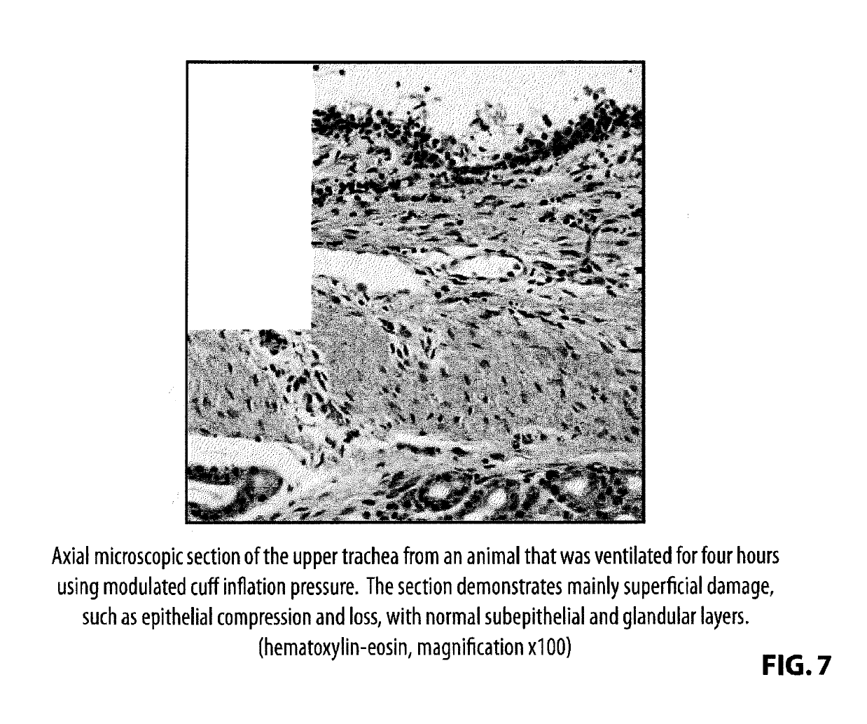

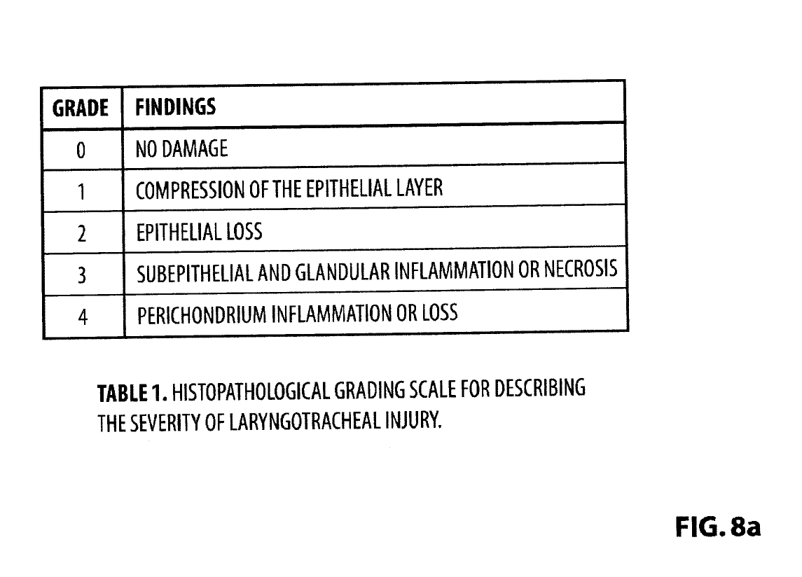

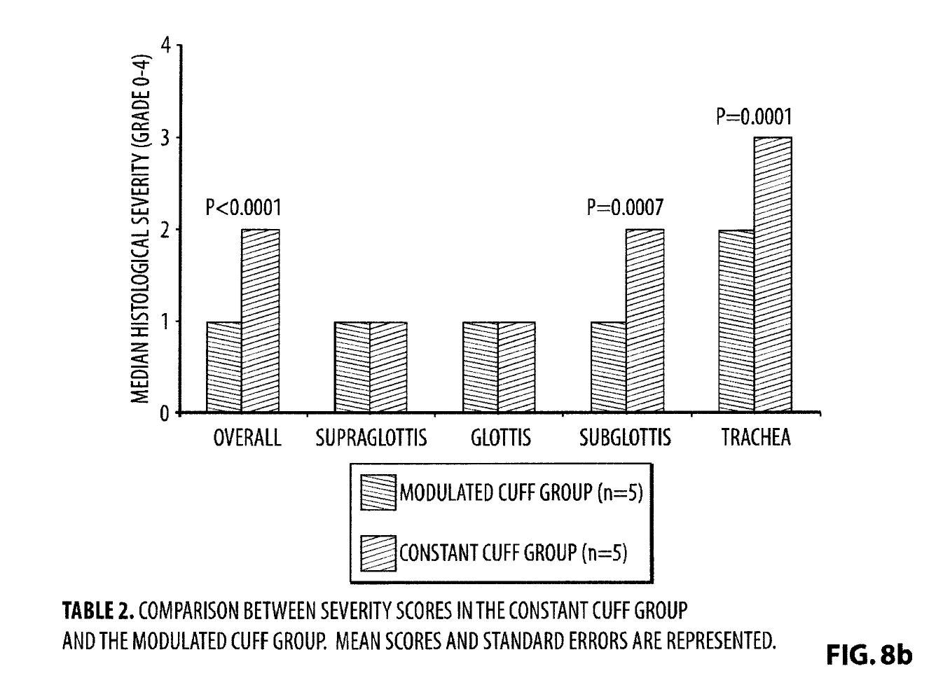

As best seen in FIGS. 4 and 11, upon expiration the expiratory valve retainer 3 sits pushed up against PEEP-like valve retainer 6. When the expiratory pressure exceeds the circuit pressure by the opening pressure of the expiratory valve, the expiratory valve disc 30 lifts off the expiratory valve retainer 3 allowing the subject to exhale. Any PEEP applied by a ventilator or anesthetic machine is added to the tracheal lumen 100 and the cuff 12. The pressure across the expiratory valve (which is dependent on the stiffness of the material of which the expiratory valve disc 30 is composed) determines the difference between the tracheal lumen pressure, alternatively called the patient airway pressure, and the cuff pressure. This difference in cuff and airway pressures is titrated to prevent fluid from passing around the cuff and into the lungs during exhalation. When PEEP is supplied by the ventilator (usually at least 3-5 cm of water), this pressure provides a positive pressure gradient between the lungs and the pharynx preventing flow of fluid into the lung. Only a slight differential increase in cuff pressure relative to hydrostatic pressure of accumulated fluid in trachea (2 to 3 cm water) is sufficient to provide protection from aspiration. As a result, during exhalation, the pressure on the mucosa by the cuff need not be much greater than 2-3 cm of H₂O to prevent aspiration.

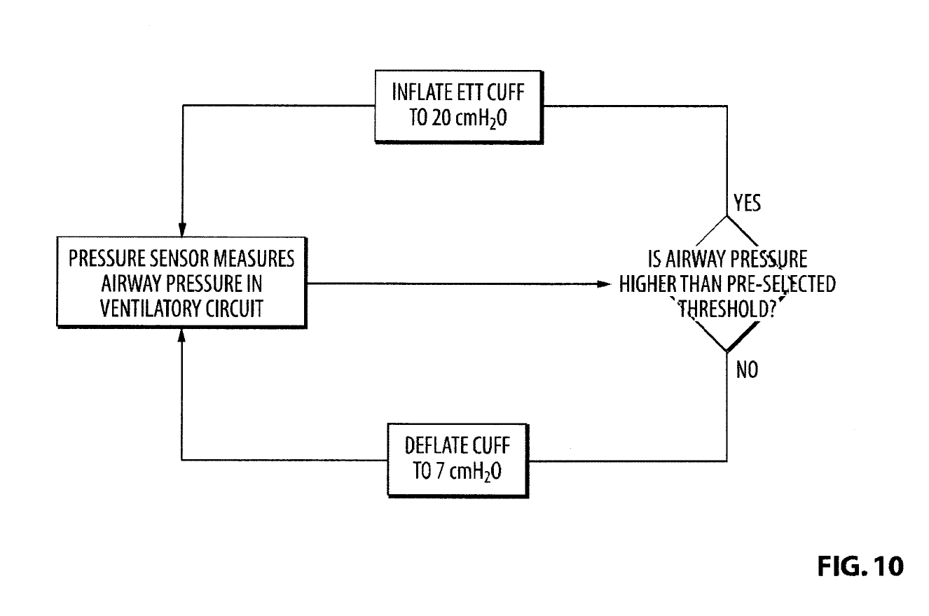

As seen in FIG. 10, the method of the invention can be accomplished with a variety of alternative more complex control circuits, including an electronic controller programmed to control pressure based on a sensor readings. This may involve measuring the pressure in the airway of the ventilator circuit or otherwise determining pressure values generated by the ventilator and then either inflating the cuff to an inspiratory cuff pressure e.g. 20 cm of water, or to a pre-selected lower expiratory cycle pressure i.e. when the ventilator pressure setting is geared to the expiratory phase of breathing, to prevent injury to the tracheal mucosa

As seen in FIG. 12, alternate devices 10 and 10a (described above) may be utilized in association with other elements of a ventilator breathing circuit used for intubation. The alternative 10b contemplates that the use of a pressure difference generator may be contribute less to benefits of preventing tracheal injury and aspiration where the selectable ventilator pressures result in higher cuff pressures since tracheal injury occurs at pressure higher the range of pressures normally used to provide PEEP and the benefits of friction may be greater at higher pressures or using different cuff materials.

### Example 1

### Summary

PATIENTS: Ten piglets (16-20 kg) were anesthetized and intubated using a cuffed endotracheal tube.

INTERVENTIONS: The animals were randomized into two groups: 5 pigs had a novel device to modulate their cuff pressure between 25 cm H₂O during inspiration and 7 cm H₂O during expiration; 5 pigs had a constant cuff pressure of 25 cm H₂O. Both groups were ventilated under hypoxic conditions for four hours.

MAIN OUTCOME MEASURES: The animals were sacrificed and the larynx and trachea harvested for blinded histopathological assessment of laryngotracheal mucosal injury.

RESULTS: The cuff pressure-modulated pigs showed significantly less laryngotracheal damage than the constant cuff pressure pigs (mean grade 1.2 versus 2.1, P<0.001). Subglottic damage and tracheal damage were significantly less severe in the modulated pressure group (mean grades 1.0 versus 2.2, P<0.001; 1.9 versus 3.2, P<0.001, respectively). There was no significant difference in glottic or supraglottic damage between the groups (P>0.05).

### Methods

The study had the full approval of the local Research Ethics Board and the Animal Care Committee. Ten female piglets, weighing 16-20 kg, were anesthetized and intubated using a cuffed endotracheal tube. The animals were randomized into two groups: in five pigs a novel device was used to modulate the cuff pressure between an maximum of 25 cm H₂O during inspiration and minimum of 7 cm H₂O during expiration (‘modulated cuff group’); the remaining five pigs had a monitored, constant cuff pressure of 25 cm H₂O (‘constant cuff group’). Both groups were ventilated for four hours under hypoxic conditions to accelerate intubation-related injury. After four hours the animals were sacrificed and the larynx and trachea were harvested for assessment by a single pathologist, who was blinded to the intervention group and study hypothesis.

### Detailed Experimental Procedure

The animals were premedicated with 0.15 ml/kg intramuscular injection of a sedative mixture (each 1 ml contained 58.82 mg ketamine, 1.18 mg acepromazine and 0.009 mg of atropine). Inhalational induction of anesthesia prior to intubation was achieved by halothane, while anesthesia thereafter was maintained with isoflurane in nitrous oxide and air/oxygen. The animals were intubated with Sheridan™ high volume, low-pressure, cuffed endotracheal tubes (Kendall-Sheridan Catheter Corporation, Argyle, N.Y.). The endotracheal tube (ETT) size was chosen by: visual inspection of the larynx; the ability to pass the tube without resistance; and the presence of a moderate air leak before cuff inflation to 25 cmH₂O. In all cases, the ETT size required was either 6.0 or 6.5 mm internal diameter. The individual performing the intubation was blinded to the study hypothesis and the intervention group. The ETT cuff pressure was measured using a cuff manometer (Posey Cufflator™, Posey, Arcadia, Calif.). Correct endotracheal tube (ETT) position was confirmed by direct visualization, auscultation, and the presence of end-tidal carbon dioxide. All intubations were successful and non-traumatic. The animals were then placed in a supine position and the ETT was secured to the snout.

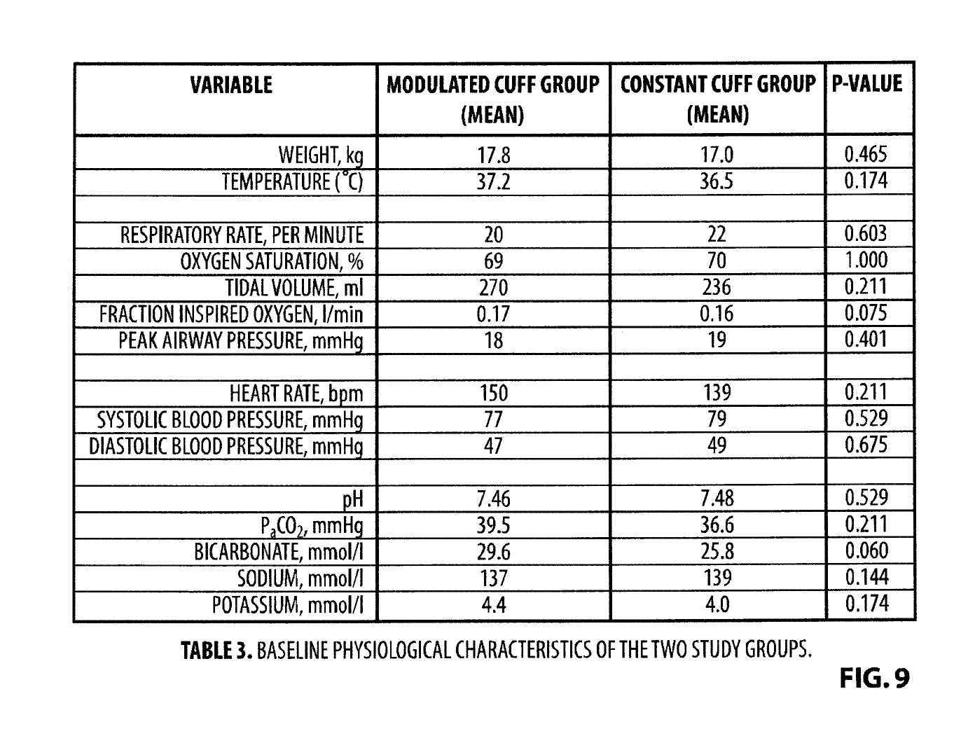

The constant cuff group had their ETT cuff pressure maintained at a constant cuff pressure of 25 cm H₂O throughout the experiment. The modulated cuff group had their cuff connected to a customized device which consisted of an in-built calibrated manometer, ventilatory pressure monitor, and a pump (see FIG. 9). This device constantly inflated and deflated the ETT cuff with each ventilatory cycle, between a maximum of 25 cm H₂O during inspiration and a minimum 7 cm H₂O during expiration. This automated device was therefore dynamically modulating the cuff pressure with a periodicity precisely synchronized with the ventilatory cycle.

Ventilation was maintained using an Air Shields Ventimeter™ volume-cycled ventilator (Narco Health Company, Pennsylvania). The right auricular vein was cannulated for intravenous fluid and drug administration. The animals were paralyzed by intravenous injection of pancuronium (bolus dose of 0.2 mg/kg and a maintenance dose of 0.2 mg/kg/hr) to prevent any ETT movements during the procedure. The left carotid artery was cannulated for invasive blood pressure monitoring and hourly arterial blood gas sampling (ABG).

The monitoring used during the experiment included heart rate, systolic and diastolic blood pressure, electrocardiography, fraction of inspired oxygen concentration (FᵢO₂), oxygen saturation, end-tidal carbon dioxide concentration and body temperature (rectal). Hypoxia was achieved by ventilating with a mixture of air and nitrous oxide. The relative concentration of air and nitric oxide were adjusted to maintain oxygen saturation between 60 and 80%, with the lowest accepted level defined as adequate ventilation without compromising the hemodynamic stability of the animal. The animals were mechanically ventilated for a total of 4 hours.

The animals were then sacrificed by a lethal intravenous injection of sodium pentobarbital (25 mg/kg). The larynx and the trachea were immediately harvested post mortem using a midline incision. The specimen was prepared for pathological assessment by an experienced pathology technician blinded to the intervention and study hypothesis. Serial axial and longitudinal sections were prepared to allow analysis of the supraglottic larynx from level of the epiglottis to the upper edge of the arytenoids), the glottis, the subglottis (immediately below the glottis to the first tracheal ring), and the upper trachea.

### Histological Evaluation

All histological evaluations were conducted by a single senior pathologist who was blinded to intervention and study hypothesis. The fixed specimens were evaluated for the severity of tissue damage. A previously described laryngeal injury grading system was employed which provided a severity grade from 0 (normal) to 4 (perichondrium involvement (see Table 1). For any given section, the severity was determined as the most severe grade of damage seen in that section.

### Statistical Analysis

The statistical methods employed for data analysis were determined a priori, using alpha=0.05 for exploring the statistical significance. Overall severity and overall extent of histological damage (using the described grading systems) were compared between the modulated cuff group and the constant cuff group using the Mann Whitney U test. Subgroup analysis was performed to compare severity between the two groups at each histological section level (supraglottic, glottic, subglottic, and trachea), using the Mann Whitney U test.

### Results

All ten animals completed the four hour intubation protocol and were included in the data analysis. The baseline characteristics of the animals and the physiologic and biochemical parameters measured during the experiment are summarized in Table 2. There was no significant difference in the baseline parameters between the modulated cuff and constant cuff groups.

The average severity scores for each group are compared in FIG. 1. Overall, the cuff pressure-modulated group had significantly less laryngotracheal histological damage than the constant cuff pressure group (mean grade 1.2 versus 2.1, p<0.001). After subgroup analysis by section level, subglottic damage and tracheal damage were found to be significantly less severe in the modulated cuff group than the constant cuff group (mean grades 1.0 versus 2.2, p<0.001; 1.9 versus 3.2, p<0.001, respectively).

1-42. (canceled)

43. A device for mitigating endotracheal tube related laryngotracheal injury associated with intubating a patient, and preventing the aspiration into the trachea and lung of potentially infected secretions from the oropharynx to prevent lung infection, the device adapted for use with a mechanical ventilator, and an endotracheal tube of the type having an inflatable endotracheal cuff, the device comprising:
a ventilator port;
an endotracheal tube port;
an air conduit portion fluidly connected to the ventilator port and the endotracheal tube port, the air conduit portion defining at least one airflow path between the ventilator port and the endotracheal tube port;
at least one pressure difference generator operatively associated with the air conduit portion, the pressure difference generator generating, in operation of the device, a pressure difference between a first pressure region of the airflow path on a ventilator side of the pressure difference generator and a second air pressure region of the airflow path on an endotracheal tube side of the pressure difference generator;
a cuff port for fluidly connecting the first pressure region and the interior of the cuff such that a first pressure in the first pressure region of the at least one airflow path substantially determines the air pressure in the interior of the cuff including a reduced cuff pressure corresponding to a ventilator pressure set for an expiratory phase of a breath.

44. A device according to claim 43, wherein the pressure difference generated by the at least one pressure difference generator determines, in cooperation with a selectable ventilator pressure setting, relative first and second pressures in the first and second pressure regions, respectively, and wherein the first pressure and the second pressure cooperate to inhibit fluid movement around the outside of the cuff when the cuff is inflated to respective differing first pressures corresponding to selectable ventilator pressure settings for an inspiratory phase of a breath and an expiratory phase of the breath, respectively.

45. A device according to claim 44, wherein the pressure difference generator is an airflow resistance element.

46. A device according to claim 44, wherein the pressure difference generator comprises a valve that opens toward the endotracheal tube at a first pressure in the first pressure region which exceeds a minimum valve opening pressure.

47. A device according to claim 46, wherein the pressure difference generator includes a valve that opens toward the ventilator at a predetermined pressure in response to a second pressure in the second pressure region.

48. A device according to claim 43, wherein the air conduit portion defines two airflow paths between the ventilator port and the endotracheal tube port and wherein a valve operatively associated with one airflow path opens toward the endotracheal tube at a predetermined minimum opening pressure that defines a first pressure region relative to a second pressure region during an inspiratory phase of a breath and wherein a valve that opens toward the ventilator at a predetermined pressure in response to a second pressure in the second pressure region is operatively associated with the other airflow path.

49. A device according to claim 44, wherein the pressure difference generator is a bi-directional valve including a first closure assembly that opens responsive to exhalation pressure generated in the second pressure region during an expiratory phase of a breath and a second closure assembly that generates a lower pressure in the second pressure region relative to a first pressure in the first pressure region during the inspiratory phase of a breath.

50. A device according to claim 43, wherein the first pressure in an inspiratory phase of breath corresponds to a cuff pressure which exceeds the second pressure (the airway pressure) by an amount sufficient to prevent substantial fluid leakage around the cuff.

51. A device according to claim 43, wherein the first pressure in an expiratory phase of breath corresponds to a cuff pressure which prevents fluid leakage around the cuff.

52. A device according to claim 44, wherein the first pressure in an expiratory phase of breath is between 1 and 5 cm of water.

53. A device according to claim 44, wherein the first pressure in an expiratory phase of breath is between 2 and 4 cm of water.

54. A device according to claim 44, wherein the first pressure in an expiratory phase of breath is between 5 and 20 cm of water.

55. A device according to claim 44, wherein the pressure difference generator comprises an expiratory valve having an opening pressure that generates positive end expiratory pressure (PEEP).

56. A method for mitigating endotracheal tube related laryngotracheal injury associated with intubating a patient with an endotracheal tube of the type having an inflatable cuff, the method comprising the step of reducing cuff pressure against the laryngotracheal mucosa to at least between 1 and 5 cm H₂O during an expiratory phase of the patient's breathing cycles by setting the PEEP setting on the ventilator to at least between 1 and 5 cm H₂O.

57. A method according to claim 56, wherein the cuff pressure against the laryngotracheal mucosa during an expiratory phase of the patient's breathing cycles is substantially determined by a ventilator pressure setting set for the expiratory phase of the patient's breathing cycles.

58. A method according to claim 56, wherein the cuff pressure is equilibrated with the ventilator pressure setting by organizing the airflow to the cuff to be channeled to the cuff from an airflow path between the ventilator and the endotracheal tube, the airflow path fluidly connected to the interior of the cuff via a cuff port.

59. A method according to claim 56 wherein the cuff pressure is organized to be different than the patient's airway pressure during an inspiratory phase of the patient's breathing cycles.

60. A method according to claim 57, wherein the cuff pressure is organized to be different than the patient's airway pressure during an expiratory phase of the patient's breathing cycles.

61. A method according to claim 56, wherein the patient's airway pressure is organized to be less the cuff pressure by interposing a pressure difference generator in the airflow path between the endotracheal tube and the cuff port, the pressure difference generator at least transiently generating a pressure difference between a first pressure region of the airflow path on a ventilator side of the pressure difference generator and a second air pressure region of the airflow path on an endotracheal tube side of pressure difference generator.

62. A method according to claim 56, wherein the pressure difference generated by the at least one pressure difference generator determines, in cooperation with a selectable ventilator pressure setting, relative first and second pressures in the first and second pressure regions of the airflow path, respectively, and wherein the first pressure and the second pressure cooperate to inhibit fluid movement around the outside of the cuff when the cuff is inflated to respective differing first pressures corresponding to selectable ventilator pressure settings for an inspiratory phase of a breath and a the expiratory phase of the breath, respectively.

63. A method according to claim 56, wherein the cuff pressure is organized to be higher than the patient's airway pressure during an inspiratory phase of the patient's breathing cycles by interposing a pressure difference generator in the airflow path between the endotracheal tube and the cuff port.

64. A method according to claim 63, wherein the pressure difference generator is a valve and wherein the opening pressure of the valve is selected from a range of 1 to 5 cm of water.

65. A method according to claim 63, wherein the minimum opening pressure of the valve is 5 cm of water and no greater than 20 cm of water.

66. The use of a device according to claim 43, for mitigating endotracheal tube related laryngotracheal injury associated with intubating a patient the use and optionally preventing the aspiration into the trachea and lung of potentially infected secretions from the oropharynx to prevent lung infection, comprising selecting a selectable ventilator setting to provide suitable inspiratory and expiratory pressures, and wherein expiratory pressure is selected to prevent tracheal injury.

67. The use according to claim 66, wherein the selectable ventilator setting for the expiratory phase of a breath for preventing laryngotracheal injury during intubation and preventing the aspiration into the trachea and lung of potentially infected secretions from the oropharynx to prevent lung infection is greater than 2 cm of water and less than 20 cm water, optionally the selected ventilator setting for the expiratory phase of a breath is 3 to 15 cm H₂O.
